(19) United States
(12) Patent Application Publication
(10) Pub. No.: US 2012/0189558 A1
(43) Pub. Date: Jul. 26, 2012

(54) ANTI-BACTERIAL COMPOSITIONS COMPRISING EXTRACTS OF EREMOPHILA LONGIFOLIA AND METHODS FOR USE OF SAME

(76) Inventor: Patrick T. Prendergast, Byrock (AU)

(21) Appl. No.: 13/393,296

(22) PCT Filed: Aug. 31, 2010
(86) PCT No.: PCT/EP10/62774

§ 371 Date: Apr. 11, 2012

(30) Foreign Application Priority Data
Aug. 31, 2009 (IE) 2009/0662

(51) Int. Cl. A61K 36/80; A61K 9/68; A01N 65/08; A61P 31/04; A61P 29/00; A61P 19/02; A61P 25/14; A61P 17/12; A61P 7/04; A01P 1/00; A61P 31/12; A61Q 11/00; A61P 1/02; A61P 13/12; A61P 25/00; A61P 43/00; A61P 27/02; A61K 8/97

(52) U.S. Cl. 424/48; 424/725; 424/58; 424/779; 424/778; 424/777; 424/773; 424/774

(57) ABSTRACT

A method for inhibiting growth of bacteria is provided wherein the method comprises the step of administering to a region in need of bacterial growth inhibition an anti-bacterially effective amount of a composition comprising an extract from the plant Eremophila longifolia. The method has utility in the treatment of a number of conditions, including cariogenesis, halitosis, gingivitis and/or periodontitis. The method of the invention may also be used ex vivo to prevent growth of bacterial biofilms on the surfaces of medical devices and other surfaces, such as those found in water systems, ventilation systems, plumbing systems, air conditioners, humidifiers and hot tubs.

### FIELD OF THE INVENTION

The present invention relates to an anti-bacterial composition and methods for use of same. More specifically, the invention relates to a composition for use in inhibiting bacteria, such as Staphylococcus aureus, Staphylococcus epidermidis, Streptococcus sobrinus, Streptococcus mutans, Streptococcus pneumoniae, Streptococcus pyogenes, Serratia marcescens, Pseudomonas aeruginosa, Stenotrophomonas maltophilia, Legionella pneumophila and Burkholderia cepacia. The invention further relates to methods for treating and preventing conditions associated with one or more of these bacteria, such as cariogenesis, halitosis, gingivitis, periodontitis and Legionnaires' disease. Also provided are ex vivo methods for inhibiting formation of bacterial biofilms on surfaces, for example, the surfaces of medical devices and other surfaces, such as those found in water systems, ventilation systems, plumbing systems, air conditioners, humidifiers and hot tubs.

### BACKGROUND TO THE INVENTION

There are many varieties of bacteria that inhabit the body of mammals. Some are “good” bacteria that help the body perform various functions, such as aiding in the digestion of food in the gastrointestinal system. Others are “bad” bacteria that cause infections, diseases and other disorders, especially in the gastrointestinal tract and respiratory system.

In the oral cavity, “bad” bacteria are responsible for, among other things, cariogenesis, halitosis, gingivitis, and periodontitis. Bad breath, which affects tens of millions of people, can be attributed to a variety of causes, including eating odiferous foods, poor oral hygiene, throat infections and tooth decay. Halitosis is a condition of chronic bad breath. While more frequent flossing and brushing of the teeth, gums, cheeks, and tongue can help reduce the problem by eliminating food particles which cause bad breath, this does not solve the problem in all cases. In many cases, bad breath can be traced to bacteria in the mouth and the toxins which they produce.

Dental caries (cariogenesis) is an infectious disease that results in irreversible damage to the tooth and the formation of cavities. The disease is known to be associated with bacteria colonising within dental plaque, with Streptococcus sobrinus and especially Streptococcus mutans being the most cariogenic pathogens. These gram positive bacteria are natural inhabitants of oral plaque that are both aciduric (acid-tolerant) and acidogenic (produce acid). Both metabolise dietary sucrose to lactic acid which causes demineralisation of the tooth's enamel and dentin and leads to a carious lesion. S. mutans is capable of synthesising sticky extracellular polysaccharides from sucrose, which is an important feature in the pathology of dental caries as it aids in their attachment to teeth forming biofilms. Biofilm-associated bacteria are more capable of tolerating changes in pH, nutrients, oxygen and the presence of antimicrobial agents. Hence, any study into a prospective naturally derived treatment for dental caries must take into consideration the structure and function of the dental biofilm environment. The growth and metabolism of S. mutans changes local environment conditions (e.g. pH) allowing the growth of more fastidious organisms forming dental plaque. Inhibition of S. mutans would therefore also be advantageous as it would also inhibit growth of these more fastidious organisms.

Gingivitis is inflammation of the gums which, if left untreated, can lead to periodontitis, in which the inflammation spreads from the gums to the ligaments and bones in the mouth. Gingivitis and periodontitis are caused by plaque deposits. Plaque is a sticky material that develops on the exposed portions of the teeth, consisting of bacteria, mucus, and food debris. Bacteria and the toxins they produce cause the gums to become infected, swollen, and tender.

Many tools and chemicals have been developed for the treatment of cariogenesis, halitosis, gingivitis and periodontitis. However, many are not effective, and others are very expensive or complicated. Accordingly, there is a need for the development of methods and treatments which can reduce or eliminate cariogenesis, halitosis, gingivitis and periodontitis. Preferably, such treatments will be simple, cost-effective and natural.

Staphylococcus epidermidis is involved in the formation of biofilms and in the conditions of endocarditis and sepsis. Staphylococcus aureus is involved in MRSA, skin infections, impetigo, cellulitis folliculitis, scalded skin syndrome (Ritters Disease), pneumonia, meningitis, osteomyelitis, endocarditis, toxic shock syndrome, sepsis and mastitis in cows.

Streptococcus pneumoniae is involved in pneumonia, acute sinusitis, otitis media, meningitis, bacteremia, sepsis, osteomyelitis, septic arthritis, endocarditis, peritonitis, pericarditis, cellulitis and brain abscess. Streptococcus pyogenes is involved in Pharyngitis, impetigo, erysipelas, cellulitis, necrotizing fasciitis, toxic shock syndrome, rheumatic fever, glomerulonephritis, obsessive compulsive disorder (OCD) and tic disorders.

Serratia marcescens has a role in urinary tract infections (UTI's), respiratory tract infections (RTI's), conjunctivitis, keratitis, endophthalmitis, tear duct infections, endocarditis, osteomyelitis, pneumonia, meningitis, teeth staining, white pox disease and viral flacherie disease. These bacteria are found on the subgingival biofilm of teeth and can cause staining. They are resistant to several antibiotics due to r-factors. They also cause white pox disease in elkhorn coral and they are a secondary pathogen in viral flacherie disease in silk worms and infect drosophila larvae and pupae in research labs.

Pseudomonas aeruginosa has a role in biofilms, pneumonia, septicaemia, UTI's, necrotising enterocolitis, haemorrhage and necrosis in burn/wound patients, hot tub rash. These bacteria also infect arabidopsis thaliana (thale cress), Lactuca sativa (lettuce), C. elegans, drosophila and galleria mellonella.

Stenotrophomonas maltophilia has a role in biofilms, pneumonia, UTI's and blood stream infections in immunocompromised patients and cystic fibrosis. They are naturally resistant to many broad spectrum antibiotics and difficult to eradicate.

Burkholderia cepacia is involved in pneumonia in immunocompromised patients.

Legionella pneumophila causes Legionnaires' disease.

The growth of bacteria is also a problem ex vivo where bacterial biofilms may form on surfaces, for example, the surfaces of medical devices and other surfaces, particularly in aquatic environments. In some cases where the medical devices are intended for use in the human body, this results in a high risk of infection. Inhibition of the growth of bacterial biofilms is therefore desirable.

The plant Eremophila longifolia is native to Australia and able to withstand extreme climates. It is also known as Emubush, Berrigan or Native plum. It is a traditional Aboriginal medicinal plant used by Aborigines externally for sores and internally as a cure for colds.

### SUMMARY OF THE INVENTION

The present invention relates to the discovery that extracts from Eremophila longifolia have anti-bacterial properties and are useful in the treatment of a number of conditions which are associated with bacteria.

Accordingly, according to a first aspect of the present invention there is provided a method for inhibiting growth of bacteria, the method comprising the step of:
administering to a region in need of bacterial growth inhibition an anti-bacterially effective amount of a composition comprising an extract from the plant Eremophila longifolia.

According to a second aspect of the present invention there is provided a composition comprising an extract from the plant Eremophila longifolia for use in inhibiting bacteria.

According to a third aspect of the present invention there is provided a method for the treatment and/or prophylaxis of cariogenesis, halitosis, gingivitis and periodontitis in a mammal, the method comprising the steps of:
providing a therapeutically effective amount of a composition comprising an extract from the plant Eremophila longifolia; and
administering the composition to the mammal.

According to a fourth aspect of the invention there is provided a composition comprising an extract from the plant Eremophila longifolia for use in the treatment and/or prophylaxis of cariogenesis, halitosis, gingivitis and periodontitis in a mammal.

According to a fifth aspect of the invention there is provided use of a composition comprising an extract from the plant Eremophila longifolia in the preparation of a medicament for the treatment and/or prophylaxis of cariogenesis, halitosis, gingivitis and periodontitis in a mammal.

According to a sixth aspect of the invention there is provided a method for the treatment and/or prophylaxis in a subject of one or more of the conditions selected from the group consisting of Legionnaires' disease, sepsis, endocarditis, skin infections, impetigo, cellulitis folliculitis, scalded skin syndrome (Ritters Disease), pneumonia, meningitis, osteomyelitis, toxic shock syndrome, mastitis, acute sinusitis, otitis media, bacteremia, septic arthritis, peritonitis, pericarditis, brain abscess, Pharyngitis, erysipelas, cellulitis, necrotizing fasciitis, rheumatic fever, glomerulonephritis, obsessive compulsive disorder, tic disorders, urinary tract infections, respiratory tract infections, conjunctivitis, keratitis, endophthalmitis, tear duct infections, teeth staining, white pox disease, viral flacherie disease, Septicaemia, necrotising enterocolitis, haemorrhage and necrosis, hot tub rash, blood stream infections and cystic fibrosis, wherein the method comprises the steps of:
providing a therapeutically effective amount of a composition comprising an extract from the plant Eremophila longifolia; and
administering the composition to the subject.

According to a seventh aspect of the present invention there is provided a composition comprising an extract from the plant Eremophila longifolia for use in the treatment and/or prophylaxis of one or more of the conditions selected from the group consisting of Legionnaires' disease, sepsis, endocarditis, skin infections, impetigo, cellulitis folliculitis, scalded skin syndrome (Ritters Disease), pneumonia, meningitis, osteomyelitis, toxic shock syndrome, mastitis, acute sinusitis, otitis media, bacteremia, septic arthritis, peritonitis, pericarditis, brain abscess, Pharyngitis, erysipelas, cellulitis, necrotizing fasciitis, rheumatic fever, glomerulonephritis, obsessive compulsive disorder, tic disorders, urinary tract infections, respiratory tract infections, conjunctivitis, keratitis, endophthalmitis, tear duct infections, teeth staining, white pox disease, viral flacherie disease, Septicaemia, necrotising enterocolitis, haemorrhage and necrosis, hot tub rash, blood stream infections and cystic fibrosis.

According to an eighth aspect of the present invention there is provided use of a composition comprising an extract from the plant Eremophila longifolia in the preparation of a medicament for the treatment and/or prophylaxis of one or more of the conditions selected from the group consisting of Legionnaires' disease, sepsis, endocarditis, skin infections, impetigo, cellulitis folliculitis, scalded skin syndrome (Ritters Disease), pneumonia, meningitis, osteomyelitis, toxic shock syndrome, mastitis, acute sinusitis, otitis media, bacteremia, septic arthritis, peritonitis, pericarditis, brain abscess, Pharyngitis, erysipelas, cellulitis, necrotizing fasciitis, rheumatic fever, glomerulonephritis, obsessive compulsive disorder, tic disorders, urinary tract infections, respiratory tract infections, conjunctivitis, keratitis, endophthalmitis, tear duct infections, teeth staining, white pox disease, viral flacherie disease, Septicaemia, necrotising enterocolitis, haemorrhage and necrosis, hot tub rash, blood stream infections and cystic fibrosis.

According to a ninth aspect of the present invention there is provided a composition comprising an extract from the plant Eremophila longifolia for use in reducing formation of lactic acid in an oral cavity.

According to a tenth aspect of the present invention there is provided a method for reducing formation of lactic acid in an oral cavity of a mammal, said method comprising the step of:
providing a therapeutically effective amount of a composition comprising an extract from the plant Eremophila longifolia; and
administering the composition to the mammal.

According to an eleventh aspect of the present invention, there is provided the use of a plant extract in a cosmetic agent for the treatment and/or prophylaxis of halitosis, characterised in that the plant extract is an extract of Eremophila longifolia.

According to a twelfth aspect of the invention there is provided a method for inhibiting the growth of a bacterial biofilm on a surface, said method comprising the step of:
administering to the surface an antibacterially effective amount of a composition comprising an extract from the plant Eremophila longifolia.

According to a thirteenth aspect of the present invention there is provided a composition comprising an extract from the plant Eremophila longifolia for use in inhibiting the growth of a bacterial biofilm on a surface.

According to a further aspect of the invention there is provided a pharmaceutical composition comprising an extract from the plant Eremophila longifolia and at least one pharmaceutically acceptable diluent, carrier or excipient.

### DESCRIPTION OF THE FIGURES

The present invention will now be described with reference to the following examples which are provided for the purpose of illustration and are not intended to be construed as being limiting on the present invention, and further with reference to the figures as described briefly below.

FIG. 1 shows a time-kill assay for E. longifolia stem extract against S. mutans. Viable cell counts at T=0, 1 and 2 hours represent the mean value of duplicate experiments (N=2, SD not shown). A sample from the same S. mutans culture was incubated without addition of stem extract to observe a control growth curve;

FIG. 2 shows time-kill assay for E. longifolia stem extract against S. sobrinus. Viable cell counts at T=0, 1 and 2 hours represent the mean value of duplicate experiments (N=2, SD not shown);

FIG. 3 shows a pH assay for E. longifolia stem extract against S. mutans. pH values at 5 minute intervals represent the mean value of duplicate experiments (N=2, SD not shown);

FIG. 4 shows the results of a pH assay for E. longifolia stem extract against S. sobrinus. pH values at 5 minute intervals represent the mean value of duplicate experiments (N=2, SD not shown);

FIG. 5 shows the results of a viable cell count performed following incubation of saliva samples for 1 hour with the three treatments and control. Values represent the mean value of duplicate experiments (N=2, SD not shown);

FIG. 6 shows the results of a salivary bacteria artificial biofilm assay. A viable count was performed to determine if the extract could affect salivary bacteria in a biofilm. Values are presented as a reduction in viable cells as compared with the water treatment. Values represent the mean value of duplicate experiments (N=2, SD not shown);

FIG. 7 shows the results of a S. mutans artificial biofilm assay. A viable count was performed to determine if the extract could affect an S. mutans biofilm. Values are presented as a reduction in viable cells as compared with the water treatment. Values represent the mean value of duplicate experiments (N=2, SD not shown);

FIG. 8 shows an SEM image of bacterial biofilms on 0.22 μm membrane filter. (a) Cells from the saliva sample showing the presence of an extracellular substance (indicated by arrows), ×15,000 magnification, (b) A cluster of S. mutans cells, ×10,000 magnification; and

FIG. 9 shows the results of a viable count performed to determine if the extract could reduce the formation of an S. mutans biofilm on 0.22 μm membrane filters. Values are presented as a reduction in viable cells as compared with the “no treatment” control. Values represent the mean value of duplicate experiments (N=2, SD not shown).

### DETAILED DESCRIPTION OF THE INVENTION

The present invention is directed to the use of an extract of Eremophila longifolia as an anti-bacterial agent and extends to its use in the treatment of conditions associated with bacteria. The methods of the present invention provide an anti-bacterial treatment which can be provided at a low cost due in part to the abundance of the Eremophila longifolia plant. Furthermore, the methods of treatment of the present invention utilise natural products which are effective and safe to the environment and the user.

In certain embodiments of the aspects of the invention the extract inhibits growth of microorganisms, in particular bacteria. In certain embodiments the bacteria comprise gram-positive and/or gram-negative bacteria. In certain embodiments the bacteria comprise cocci such as Streptococcus and/or Staphylococcus. In certain embodiments the bacteria comprise Serratia, Pseudomonas, Stenotrophomonas and/or Burkholderia. In certain embodiments the bacteria comprise at least one of the bacteria selected from the group consisting of Staphylococcus aureus, Staphylococcus epidermidis, Streptococcus sobrinus, Streptococcus mutans, Streptococcus pneumoniae, Streptococcus pyogenes, Serratia marcescens, Pseudomonas aeruginosa, Stenotrophomonas maltophilia and Burkholderia cepacia. In certain embodiments, the bacteria comprise MRSA. In certain embodiments, the bacteria comprise Legionella pneumophila.

In certain embodiments the composition comprising the extract is administered to a mammal, such as a human. The composition may be administered to any region in need of bacterial growth inhibition. In certain embodiments the composition is administered to an oral cavity of a mammal, for example, the oral cavity of a human. In alternative embodiments the composition is administered to the skin.

In certain embodiments the composition is used in the treatment and/or prevention of conditions associated with Streptococcus sobrinus and/or Streptococcus mutans, for example, cariogenesis, halitosis, gingivitis and periodontitis. In certain embodiments the composition is used in the treatment and/or prevention of cariogenesis. Streptococcus sobrinus and/or Streptococcus mutans play a major role in tooth decay. Accordingly, inhibition of one or both of these bacteria may be used to prevent tooth decay. In particular, inhibition of Streptococcus mutans may be used to prevent formation of dental plaque. Inhibition of Streptococcus mutans inhibits changes in the local environmental conditions (e.g. pH), thereby inhibiting growth of other organisms which depend on these changes in conditions. In particular, inhibition of Streptococcus mutans reduces the formation of acid, such as lactic acid. Reducing the development of lactic acid can avoid weakening of enamel on teeth. Inhibition of Streptococcus sobrinus also reduces the formation of acid, such as lactic acid.

In certain embodiments the composition is used in the treatment and/or prophylaxis of conditions associated with Staphylococcus epidermidis, for example, sepsis, endocarditis and bacterial biofilms.

In certain embodiments the composition is used in the treatment and/or prophylaxis of conditions associated with Staphylococcus aureus, for example, sepsis, skin infections, impetigo, cellulitis folliculitis, scalded skin syndrome (Ritters Disease), pneumonia, meningitis, osteomyelitis, endocarditis, toxic shock syndrome and/or mastitis. The composition may also be used in the treatment and/or prophylaxis of MRSA.

In certain embodiments the composition is used in the treatment and/or prophylaxis of conditions associated with Streptococcus pneumoniae, for example, pneumonia, acute sinusitis, otitis media, meningitis, bacteremia, sepsis, osteomyelitis, septic arthritis, endocarditis, peritonitis, pericarditis, cellulitis and brain abscess.

In certain embodiments the composition is used in the treatment and/or prophylaxis of conditions associated with Streptococcus pyogenes, for example, Pharyngitis, impetigo, erysipelas, cellulitis, necrotizing fasciitis, toxic shock syndrome, rheumatic fever, glomerulonephritis, OCD and tic disorders.

In certain embodiments the composition is used in the treatment and/or prophylaxis of conditions associated with Serratia marcescens, for example, UTI's, RTI's, conjunctivitis, keratitis, endophthalmitis, tear duct infections, endocarditis, osteomyelitis, pneumonia, meningitis, teeth staining, white pox disease and viral flacherie disease.

In certain embodiments the composition is used in the treatment and/or prophylaxis of conditions associated with Pseudomonas aeruginosa, for example, biofilms, Pneumonia, Septicaemia, UTI's, necrotising enterocolitis, haemorrhage and necrosis in burn/wound patients, hot tub rash.

In certain embodiments the composition is used in the treatment and/or prophylaxis of conditions associated with Stenotrophomonas maltophilia, for example, biofilms, Pneumonia, UTI's and blood stream infections in immunocompromised patients and cystic fibrosis.

In certain embodiments the composition is used in the treatment and/or prophylaxis of conditions associated with Burkholderia cepacia, for example, pneumonia in immunocompromised patients.

In certain embodiments the composition is used in the treatment and/or prevention of conditions associated with Legionella pneumophila, in particular, Legionnaires' disease. Legionella pneumophila dwells in man-made and natural aquatic environments and is spread through contaminated water or ventilation systems, plumbing systems, air conditioners, humidifiers and hot tubs. Accordingly, inhibition of Legionella pneumophila in these environments may assist in preventing the spread of Legionnaires' disease.

In certain embodiments the method of inhibiting the growth of bacteria and/or the growth of a bacterial biofilm on a surface is an in vivo method. In certain embodiments the region in need of bacterial growth inhibition or the surface is the surface of a tooth. In certain embodiments, the bacterial biofilm comprises plaque.

In certain embodiments the method of inhibiting the growth of bacteria and/or the growth of a bacterial biofilm on a surface is an ex vivo method. In certain embodiments the region in need of bacterial growth inhibition or the surface is any surface on which the formation of bacteria would be undesirable, for example, the surface of a medical device or the surface of plastics used in hospital procedures. For example, the composition may be used for disinfecting medical devices, in particular, medical devices such as those intended for use in vivo. In alternative embodiments the region in need of bacterial growth inhibition or the surface relates to a non-medical device, such as an aquatic environment, water system, ventilation system, plumbing system, air conditioner, humidifier or hot tub.

In certain embodiments the composition may be applied to the surface as an antimicrobial coating agent. Additionally or alternatively, the composition may be incorporated in a substance at the time of manufacture, for example, by coating, dipping or chemical binding, in order to make the substance at least partially resistant to colonisation by bacteria.

In certain embodiments of the method of the invention relating to the inhibition of the growth of a bacterial biofilm, the plant extract of Eremophila longifolia results in the detachment of bacterial cells from the surface. Inhibition by detachment of the cells may be advantageous in preventing the development of resistant strains (Duarte et al., 2006). Additionally and/or alternatively, the plant extract of Eremophila longifolia may kill bacterial cells.

A reduction in viable biofilm cells is important because biofilm-associated bacteria are more capable of tolerating the presence of antimicrobial agents. In certain embodiments the method of the invention includes a further step of administering a second antimicrobial agent. The second antimicrobial agent may be more effective once the bacterial cells have become detached from the surface.

In certain embodiments the plant extract is derived from the stem, leaves, roots, branches, fruit or flower of Eremophila longifolia. In certain embodiments the plant extract is derived from the stem of Eremophila longifolia. This extract has been shown to be particularly effective in the methods of the invention. In certain embodiments the composition may comprise extracts derived from two or more parts of the plant.

It is thought that E. longifolia growing in different geographical locations contain different compounds, which produce extracts having different colours and fragrances. This hypothesis has been explored and substantiated by a number of studies into a variety of plant extracts (Ozcan and Chaichat, 2005; Celiktas et al., 2007; Shene et al., 2009). TLC analysis of extracts of E. longifolia from different locations has shown very distinct differences in colour and positions of separated bands. Accordingly, the anti-bacterial effect of the extract may be increased depending upon the geographical location where the plant from which the extract is derived was cultivated. In certain embodiments the extract is from Eremophila longifolia of the type cultivated in New South Wales and/or the Northern Territory of Australia, for example, Byrock. These types have been found to have an increased anti-bacterial effect. In certain embodiments the extract is not obtained from Eremophila longifolia of the type cultivated in West Australia. Although this type has been shown to have an anti-bacterial effect, this effect has been shown to be reduced when compared to extract derived from plants cultivated in other locations.

In certain embodiments the extract is extracted from Eremophila longifolia using a solvent. In certain embodiments the solvent is selected from the group consisting of ethanol, acetone and water. In certain embodiments the solvent is ethanol. In certain embodiments the composition comprises an ethanolic extract from the plant Eremophila longifolia. The term “ethanolic extract” refers to an extract obtained from the plant using ethanol as a solvent. The extract may be re-dissolved in ethanol following evaporation of the solvent. The ethanolic extract has been shown to be particularly effective in the methods of the invention. Additional suitable extraction methods will be well known to those skilled in the art and include any conventional methods used in the field. These include, but are not limited to, solvent extraction, steam or dry distillation, cold pressing and hyperbaric extraction.

In certain embodiments references to a composition comprising an extract from the plant Eremophila longifolia extend to compositions comprising an analogue, metabolite, precursor, derivative, synthetic version, pharmaceutically active salt or pro-drug of the active agent of the extract wherein the analogue, metabolite, precursor, derivative, pharmaceutically active salt or pro-drug retains the same anti-bacterial activity as the active agent of the extract. In certain embodiments the active agent is obtained from a source other than the extract, but retains the same anti-bacterial activity as the extract. For example, in certain embodiments the active agent is synthetically derived.

In certain embodiments the active agent is selected from the group consisting of a phenolic compound, such as a flavonoid, a terpene, an alkaloid or a new molecular entity, such as a new phenolic compound or a new flavonoid. Accordingly, in certain embodiments the invention relates to a method for inhibiting growth of bacteria, the method comprising the step of:
administering to a region in need of bacterial growth inhibition an anti-bacterially effective amount of a composition comprising an active agent wherein the active agent is selected from the group consisting of a phenolic compound, such as a flavonoid, a terpene and an alkaloid and wherein the active agent retains the same anti-bacterial activity as the extract from the plant Eremophila longifolia.

In certain embodiments the composition comprises a pharmaceutically acceptable diluent, excipient or carrier. The pharmaceutically acceptable diluent, excipient or carrier may be chosen based on the intended route of administration of the resulting pharmaceutical composition. In certain embodiments, the composition is formulated in beta-hydroxycyclodextrin. In certain embodiments, the pharmaceutically acceptable carrier is selected from the group consisting of cyclodextrin, alpha-cyclodextrin, beta-cyclodextrin, (beta-hydroxypropylcyclodextrin) gamma-cyclodextrin and vitamin E oil.

In certain embodiments the methods of the invention further comprise the step of administering one or more additional anti-bacterial agents to the mammal. Suitable anti-bacterial agents will be known to persons skilled in the art. These may be administered sequentially, simultaneously or separately to the plant extract.

As used herein, the term “subject” refers generally to an animal. A “subject” in the context of the present invention therefore includes and encompasses mammals, such as humans, primates and livestock animals (e.g. sheep, pigs, cattle, cows, horses, donkeys); laboratory test animals, such as mice, rabbits, rats and guinea pigs; and companion animals, such as dogs and cats. It is preferred for the purposes of the present invention that the mammal is a human. In certain embodiments the subject is an immunocompromised subject. In certain embodiments the subject is a burn/wound patient.

In certain embodiments the composition comprising the plant extract is administered to a subject via any suitable route. In certain embodiments wherein the bacteria to be inhibited are present in the oral cavity, the composition is administered to the oral cavity. In alternative embodiments routes of administration may include, but are not limited to, parenterally (including subcutaneous, intramuscular and intravenous, by means of, for example a drip patch), oral, rectal (suppositories), nasal, gastric, topical (including buccal and sublingual), infusion, vaginal, intradermal, intraperitoneally, intracranially, intrathecal and epidural administration.

For administration via the oral routes the extract is in a suitable pharmaceutical formulation. In certain embodiments the composition comprising the plant extract is selected from the group consisting of a mouth wash, toothpaste, oral spray, oral cream or gel, candy, dissolvable pill or strip, chewing gum, lozenge and powder that can be sprinkled directly into the oral cavity. In certain embodiments the extract is delivered using a mechanical form including, but not restricted to an inhaler, nebuliser device or a nasal spray. Further, where the oral inhalation route is used, administration by a SPAG (small particulate aerosol generator) may be used. Pharmaceutical compositions for oral administration may be in tablet, solid, capsule, powder or liquid form. A tablet may comprise a solid carrier such as gelatin or an adjuvant. Liquid pharmaceutical compositions generally comprise a liquid carrier, such as water, petroleum, animal or vegetable oils, mineral oil or synthetic oil. Physiological saline solution, dextrose or other saccharide solutions or glycols such as ethylene glycol, propylene glycol or polyethylene glycol may be included. Suitable formulations for oral administration further include hard or soft gelatin capsules, dragees, pills, tablets, including soft-coated tablets, troches, lozenges, melts, powders, micronized particles, non-micronized particles, solutions, emulsions, elixirs, suspensions, syrups or inhalations and controlled release forms thereof.

In certain embodiments, administration is topical. Suitable formulations for topical administration include creams, gels, jellies, mucliages, pastes and ointments. The compounds may be formulated for transdermal administration, for example in the form of transdermal patches so as to achieve systemic administration.

The composition may also be administered via microspheres, liposomes, other microparticulate delivery systems or sustained release formulations placed in certain tissues including blood. In certain embodiments the composition may be implanted into a subject or injected using a drug delivery system.

The composition according to the present invention may be administered locally or systemically. Systemic administration is understood to refer to any mode or route of administration that results in effective amounts of extract appearing in the blood or at a site remote from the site of administration.

In certain embodiments, the extract is micronized. The term “micronized” is intended to mean that the compound has been micronized in accordance with any process for micronizing, a number of which are known in the art. The micronized particles preferably include a percentage of particles having a diameter of about 10 microns, or less, preferably 5 microns or less. For example, in a certain aspect of the invention, at least 80% of the particles in a formulation of micronized particles have a diameter of less than 5 microns.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, N. C. et al. 7th Edition ISBN 0-683305-72-7, the entire disclosures of which is herein incorporated by reference.

The actual amount administered, and the rate and time-course of administration, will depend on the nature and severity of the condition to be treated. Prescription of treatment, e.g. decisions on dosage etc., is ultimately within the responsibility and at the discretion of general practitioners and other medical doctors, and typically takes account of the condition to be treated, the condition of the individual subject, the site of delivery, the method of administration and other factors known to practitioners. The precise dose will depend upon a number of factors, including the form of the composition to be administered.

In certain embodiments the composition is administered at a concentration of at least 5 mg/ml. This concentration has been shown to be the minimum inhibitory concentration of the ethanol extract against S. mutans and S. sobrinus. In certain embodiments the composition is administered at a concentration of at least 10 mg/ml.

In alternative embodiments the composition is administered at a concentration of less than 5 mg/ml. This concentration is below the minimum inhibitory concentration of the ethanol extract against S. mutans and S. sobrinus, but has been shown to be effective in reducing the production of lactic acid.

In certain embodiments the composition is administered at a concentration of 2 mg/L or greater, preferably 4 mg/L or greater. These concentrations have been shown to be the minimum inhibitory concentrations for S. epidemidis.

In certain embodiments the composition is administered at a concentration of 8 mg/L or greater. This concentration has been shown to be the minimum inhibitory concentrations for S. aureus.

In certain embodiments the composition is administered at a concentration of 32 mg/L or greater. This concentration has been shown to be the minimum inhibitory concentrations for Stenotrophomonas maltophilia and Burkholderia cepacia.

In certain embodiments the composition is administered at a concentration of 64 mg/L or greater. This concentration has been shown to be the minimum inhibitory concentrations for Streptococcus pneumoniae, Streptococcus pyogenes, Serratia marcescens and Pseudomonas aeruginosa.

In certain embodiments the composition is administered daily to a subject. Typically, the composition may be used as part of a subject's oral care wherein the composition is administered every morning and night. In certain embodiments the composition is administered intermittently.

As used herein, the term “treatment” and associated terms such as “treat” and “treating” mean the prevention or reduction of bacterial growth or the prevention or reduction of the progression, severity and/or duration of any symptom associated with the condition being treated, wherein said reduction results from the administration of a composition of the invention. The term “treatment” refers to any regimen that can benefit a subject. The treatment may be in respect of an existing condition or may be a prophylactic (preventative) treatment. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Accordingly, therapeutic and prophylactic treatment includes amelioration of the symptoms of a particular condition or preventing or otherwise reducing the risk of developing a particular condition. The term “prophylactic” may be considered as including reducing the severity or the onset of a particular condition.

The composition comprising the extract may be administered in an “anti-bacterially effective amount”, this being an amount sufficient to at least partially inhibit or reduce activity of the bacteria, for example, growth of the bacteria or development of a bacterial biofilm. Alternatively, the composition may be administered to a subject in a “therapeutically effective amount”, this being an amount sufficient to show benefit to the subject. In particular, the benefit may be the treatment, partial treatment or amelioration of at least one symptom associated with the condition being treated, or the prevention or partial inhibition of the onset of at least one symptom associated with that condition. The severity and/or time of onset of the at least one symptom may be reduced. Where the context demands, a “therapeutically effective amount” is an amount which induces, promotes, stimulates or enhances the development of an antibacterial response by the subject.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

As used herein, terms such as “a”, “an” and “the” include singular and plural referents unless the context clearly demands otherwise.

Unless otherwise defined, all technical and scientific terms used herein have the meaning commonly understood by a person who is skilled in the art in the field of the present invention.

### Example 1

### Assessment of Anti-Bacterial Effect of Eremophila longifolia Extracts on S. mutans and S. sobrinus

### Materials and Methods

### Extraction of Plant Material

Aerial parts of Eremophila longifolia were collected from plants growing in Byrock, NSW, in November 2007. The fresh material was transported to Swinburne University of Technology and stored at −20° C. until freeze-drying.

Leaf and stem material were separated and cut into small pieces using gardening secateurs. Both samples were freeze-dried for 22 hours in a Telstar Cryodos freeze-dryer and then crushed into smaller pieces with a mortar and pestle. Three polar solvents were used for the extraction of plant material: acetone (100%), absolute ethanol (>99%) and Milli-Q distilled water. Extraction involved soaking approximately 2 g of the crushed sample in 75 ml of each solvent for 5 days at room temperature with occasional agitation. Ethanol and acetone was removed from the extracted material using a Buchi Rotavapor rotary evaporator with the water temperature set at 40° C. Water was removed from the extracted material by freeze-drying for 20 hours. The residual extract was weighed and re-dissolved in the extracting solvent at a concentration of 100 mg/ml. Extracts were stored at 4° C.

Additional ethanolic stem extract was prepared following the initial method, with the exception of an added evaporation step following rotary evaporation, as follows: the majority of solvent was removed during rotary evaporation and then the liquid was poured into a large glass petri dish and further concentrated to dryness in a vacuum desiccator for 3 hours.

### Microorganisms and Media

Extracts were tested against two Gram positive cariogenic bacteria: Streptococcus mutans (969) and S. sobrinus (6715-247). These strains were provided by the Melbourne Dental School, University of Melbourne, and are part of a culture collection located at Swinburne University of Technology.

Working cultures of S. mutans and S. sobrinus were maintained on Brain Heart Infusion (BHI) agar slopes, prepared by adding 1.5% agar (Oxoid Ltd) to BHI broth (Oxoid Ltd and Difco Ltd). For experiments, both bacteria were grown on BHI agar overnight at 37° C. in a candle jar which provided reduced oxygen conditions. When needed, liquid bacterial cultures were prepared by inoculating 3 ml of BHI broth and growing overnight at 37° C. All media were prepared in deionised water and autoclaved at 121° C. for 20 minutes prior to use.

### Plate-Hole and Disk Diffusion Assays

Plate-hole diffusion assays were used to test for antibacterial activity (Palombo and Semple, 2001). A pure colony of each culture was grown in BHI broth and 200 μL were added to 15 ml of molten BHI agar. The inoculated agar was gently mixed and transferred to a sterile petri dish. Once set and dried, a sterilised core-borer (6 mm diameter) was used to make wells in the agar, and 10 μL of plant extract were added into each well. One well on each plate was filled with neat solvent as a control. Disk diffusion assays were also used to test for antibacterial activity (Pennacchio et al., 2005). 10 μL of each extract and control were placed on sterile paper disks (6 mm diameter, Oxoid) and allowed to dry for 25 minutes. 100 μL of each overnight (ON) culture was spread on BHI agar and allowed to dry for 10 minutes. Disks were transferred to the agar surface. Both plate-hole and disk diffusion assays were incubated overnight at 37° C. in candle jars and were carried out in triplicate. A clear zone of inhibited bacterial growth surrounded substances exhibiting antibacterial properties and zones with a diameter greater than 6 mm were considered positive.

### Minimum Inhibitory Concentration (MIC) Assays

The opacity of the plant extract meant that the standard MIC assay could not be performed as it relies on the observation of turbidity of inoculated broth. The modified method used involved observing the presence of a clear zone in a plate-hole diffusion assay. Dilutions of the active extract were made in the vehicle solvent, ethanol, and 10 μL of each were transferred into wells made in BHI agar seeded with either S. mutans or S. sobrinus. Plates were incubated in candle jars at 37° C. overnight and observed for the presence of inhibition. The minimum inhibitory concentration was considered to be the lowest concentration with a visible zone of inhibition. This assay was carried out in triplicate. Due to the semi-quantitative nature of plate-hole diffusion zones, this method can only be used as an estimation of the actual MIC.

### Time-Kill Assays

BHI broth (0.5 ml) was inoculated with 0.5 ml of ON S. mutans or S. sobrinus culture. 100 μL of stem extract were added to each vial to give a final concentration of 10 mg/ml. A 100 μL aliquot was spread onto a BHI agar plate and a further 100 μL was collected to enumerate viable cells by serial dilution in sterile BHI broth (10-1 to 10-5) and immediately spread on BHI agar plates. Vials were incubated at 37° C. for 2 hours with gentle shaking, and samples were taken every hour as described above. Controls were prepared following the same method without the addition of plant extract. Plates were incubated in candle jars at 37° C. overnight and a viable count was then performed. Time-kill assays were performed in duplicate.

### pH Assay

Cells of S. mutans and S. sobrinus from suspension cultures were harvested, washed once with salt solution (50 mM KCl, 1 mM MgCl2), and re-suspended in 5 ml salt solution containing 166 μL stem extract (final concentration 3.3 mg/ml). The pH was adjusted to between 7.35-7.47 with 0.1 M KOH solution and sufficient glucose was added to give a concentration of 1% (w/v). The decrease in pH was measured every 5 minutes over a period of 30 minutes using a glass electrode (TPS). A solvent control was prepared by adding 166 μL ethanol to each bacterial system instead of stem extract and a “no treatment” control involved measurement of pH drop without addition of extract or solvent. (Duarte et al., 2006).

### Antibacterial Activity Against Salivary Bacteria

Non-stimulated saliva was collected from a healthy donor and 200 μL aliquots were transferred to four sterile microcentrifuge tubes. Stem extract was added to two tubes at a concentration of 5 mg/ml and 10 mg/ml, respectively, and chlorhexidine (J & J Medical) was added to another tube at a concentration of 2 mg/ml. All four tubes were incubated for 1 hour at 37° C. before serial dilutions were performed and 100 μL of each dilution were spread on BHI agar. Plates were incubated in candle jars for 18 hours at 37° C. and the resultant colonies were counted and recorded.

### Artificial Biofilm Assays

Artificial biofilm assays were performed based on the method of Alviano et al. (2008). Non-stimulated saliva was collected from a healthy donor and 20 μL was placed on sterile 0.22 μm Millipore membrane disks of 13 mm diameter, previously placed over BHI agar plates. Plates were incubated for 48 hours at 37° C. After biofilm growth, the disks were collected and each disk was placed inside a bottle containing 3 ml of stem extract (5 mg/ml or 10 mg/ml in ethanol), Milli-Q distilled water or ethanol for 1 hour at 37° C. with gentle shaking. Then, the disks were briefly washed with Milli-Q distilled water to remove the plant extract and unbound bacterial cells, and the biofilm was extracted by vortexing the disks in 1 ml of BHI broth. Immediately, serial dilutions were performed and 100 μL of each dilution were spread on BHI agar. The plates were incubated in candle jars for 48 hours at 37° C. and a viable count was performed. An S. mutans artificial biofilm assay was performed by repeating the above method except that a pure ON culture of S. mutans was grown on the membrane disks instead of salivary bacteria. Both this assay and the salivary assay were performed in duplicate.

### Scanning Electron Microscopy (SEM)—Biofilm Observations

Salivary bacteria biofilms and S. mutans biofilms were grown on membrane disks as described above. Disks were washed with Milli-Q distilled water to remove loosely attached bacteria and affixed to a glass slide with double-sided tape. The biofilm samples were dehydrated, coated with carbon, and spluttered with gold using a Dynavac CS300 coating unit. The samples were then visualised with a FeSEM instrument (Supra 40 VP, Carl Zeiss).

### Inhibition of Attachment

This method was based on a beaker-wire test performed by Kang et al. (2008), which evaluated S. mutans accumulation on stainless steel wire in the presence of a treatment. Stem extract (5 mg/ml and 10 mg/ml) and ethanol was added to 3 bijoux bottles containing 3 ml of BHI broth supplemented with 5% sucrose and 0.1M of 2-[N-Morpholino] ethanesulfonic acid monohydrate (MES). S. mutans was inoculated, and three nickel chromium wires attached to sterile 0.22 μm filter membranes were immersed in the system. The tubes were covered and incubated with slow agitation at 37° C. for 24 hours. The filter membranes were then removed, detached from the wire, and gently rinsed with distilled Milli-Q water and vortexed in 1 ml of BHI broth. A serial dilution and viable count was then performed to evaluate the number of bacterial cells that were able to attach to the membrane in the 24-hour time period.

### Preliminary Phytochemical Analysis—Microscale Column Chromatography

A glass Pasteur pipette was plugged with a small amount of glass wool and filled to 8 cm with dry silica gel (Labchem 100-200 mesh). Pre-elution of the column was performed with a hexane: ethanol (9:1) solvent, before addition of 150 μL of stem extract. Further mobile phase was added to the column and a pipette bulb was used intermittently to gently apply positive pressure. After approximately 40 minutes and the addition of 3.4 ml of solvent, the mobile phase was altered to hexane: ethanol, 6:4. Fractions were collected according to colour change until the elution ran clear. Finally, 100% ethanol was added to the column to elute any polar compounds bound to the silica gel. All fractions were dried in a vacuum dessicator for 2 hours, weighed, and diluted to 100 mg/ml in ethanol. All fractions were assessed for their antibacterial activity using the plate-hole diffusion method described above.

### Preliminary Phytochemical Analysis—Thin Layer Chromatography (TLC)

TLC was performed on both the crude extract and one of the extract fractions. In each case, 7 μL of sample were placed on a silica TLC plate with aluminium backing (Sigma). The TLC plates were placed in a sealed beaker containing a solvent mixture, until the solvent had been drawn up three-quarters of the length of the silica sheet. Components were separated based on relative affinity for the solvent or the silica. Plates were developed in different solvent systems—8:2, 9:1 and 10:0 toluene: ethanol—and the system providing the greatest separation was selected for bioautography analysis.

### Preliminary Phytochemical Analysis—Bioautography

Developed TLC plates were allowed to dry for 30 minutes and then placed into sterile petri dishes. For each plate, 200 μL of ON S. mutans or S. sobrinus culture were added to 15 ml of molten BHI agar, mixed and poured over the TLC plate under aseptic conditions. The agar was allowed to set, and the plates were incubated in candle jars overnight at 37° C. To improve visualisation of colonies and zones of inhibited growth, a 2% solution of methylthiazolyltetrazolium chloride (MTT) dye was sprayed on the plates, resulting in colourisation of living cells.

### Preliminary Phytochemical Analysis—Identification of Compound Groups Using Spray Reagents

Aluminium chloride is used for detection of flavonoids. 1% aluminium chloride in ethanol (Krebs et al., 1969) solution was lightly sprayed over the top of developed TLC plates and they then were viewed under ultra-violet light at 360 nm. Separated bands that contain flavonoid compounds fluoresce yellow

Dragendorff reagent is used for detection of alkaloids. 8 g of potassium iodide was dissolved in 20 ml of water. This solution was mixed with a solution containing 0.85 g bismuth subnitrate in 40 ml of water with 10 ml acetic acid. After spraying, the presence of yellow zones in visible light suggests alkaloid compounds.

Folin-Ciocalteu reagent is used for detection of phenolic compounds. After spraying with Folin-Ciocalteu reagent (Merck), plates were observed in visible light for the presence of blue zones.

Liebermann-Burchard reagent is used for detection of triterpenes, steroids and sterols. This reagent was prepared by adding 5 ml of acetic anhydride and 5 ml of concentrated sulphuric acid to 50 ml of absolute ethanol on ice. TLC plates were sprayed and then warmed at 100° C. for 10 minutes. Separated bands were evaluated for the presence of blue/green colour.

### Results

### Extraction of Plant Material

Three polar solvents were chosen for the extraction process because previous studies have suggested that polar solvents are more successful in extracting active compounds from plant material (Cowan, 1999). The dry mass of both the stem and leaf material was determined following freeze-drying, and the residual extract remaining after solvent evaporation was weighed to determine the yield of extract for each solvent.

TABLE 1
Amount of stem and leaf extract produced by soaking in different polar
solvents

| | | | |
|---|---|---|---|
| | | Amount of | |
| | Dry mass of | extract produced | |
| Solvent | plant material (g) | (g) | Yield % |

Stem material

| | | | |
|---|---|---|---|
| Water | 1.99 | 0.32 | 16.08 |
| Ethanol | 2.00 | 0.15 | 7.5 |
| Ethanol^{a} | 13.04 | 1.08 | 8.28 |
| Acetone | 2.00 | 0.09 | 4.5 |

Leaf material

| | | | |
|---|---|---|---|
| Water | 2.00 | 0.24 | 12.00 |
| Ethanol | 1.99 | 0.11 | 5.53 |
| Acetone | 2.00 | 0.08 | 4.00 |

^{a}Additional ethanolic extract of the stem material was produced at a later date, with a further evaporation step as detailed in the section entitled “Extraction of plant material”.

The general trend in the yield of extracts seen in Table 1 was an increase in yield as the polarity of the solvent increased. For both the stem and leaf material, the acetone solvent produced the lowest yield and the most polar solvent, water, produced the highest yield. Although a non-polar solvent was not included for comparison, these results suggest that both samples contain a relatively large amount of compounds with a high affinity for highly polar solvents in comparison to those with an affinity for moderately polar solvents. The extracts were re-dissolved in the same solvent that was used for their extraction, to a concentration of 100 mg/ml.

### Plate-Hole and Disk Diffusion Assays

The six extracts obtained from E. longifolia were screened for antibacterial activity against the known cariogenic bacteria, S. mutans and S. sobrinus. An assessment of antibacterial activity was made by observing the zone of inhibition produced by each extract in plate-hole and disk diffusion assays.

The antibacterial assays were performed on the neat extracts (100 mg/ml). Each agar plate included a solvent control to ensure that the solvent component within the extracts had no effect on bacterial growth. Although ethanol is often used as a disinfecting agent, it is the water component of a 70-75% ethanol solution that makes it active against the bacteria. Therefore the >99% ethanol used to re-dissolve the ethanolic extracts would not have an effect on bacterial growth. The control assays confirmed that ethanol, acetone and water did not inhibit bacterial growth.

The antibacterial activity of chlorhexidine is well documented and it was therefore used as a positive control in this study to validate test methods. Each of the six extracts and chlorhexidine were tested against the two cariogenic bacteria and the diameters of the zones of inhibition were measured (Table 2). The diameter of the agar wells and sterile disks was 6 mm; therefore zones of inhibited growth >6 mm were considered positive.

TABLE 2
Antibacterial activity of leaf and stem extracts of E. longifolia.

| | | | | Stem extract (100 mg/ml) | | | |
|---|---|---|---|---|---|---|---|
| Leaf extract (100 mg/ml) | | | | | | | Chlorhexidine |
| | Water | Ethanol | Acetone | Water | Ethanol | Acetone | (2 mg/ml) |
| S. mutans | 6.0 +/− 0 | 6.4 +/− 0.6 | 6.5 +/− 0.7 | 6.4 +/− 0.6 | 17.1 +/− 0.7 | 17.9 +/− 0.8 | 22.1 +/− 0.6 |
| S. sobrinus | 6.0 +/− 0 | 6.4 +/− 0.8 | 6.7 +/− 0.6 | 6.3 +/− 0.5 | 15.9 +/− 0.5 | 16.7 +/− 0.7 | 20.5 +/− 0.6 |

Values represent the mean diameter of the growth inhibition zone (mm)+SD, from three plate-hole assays and three disk diffusion assays.

As expected, chlorhexidine exhibited activity against both S. mutans and S. sobrinus, producing inhibition zones of 22.1+/−0.6 and 20.5+/−0.6, respectively. This antiseptic agent, at a concentration of 2% (mg/ml), is the active ingredient in range of medicated mouth rinses, including Savacol. The water extract of the stem material exhibited minimal inhibition of the cariogenic bacteria. This may have been because the active components of the stem extract are compounds not usually extracted in water, or the low temperature of the water may have not provided the kinetic energy necessary to remove the active components. If the extraction had been performed with boiling water, it is possible that the active components would have been extracted. Despite the low temperature of the water extraction, more than twice the amount of extract was produced compared with the ethanol extraction. This suggests that many E. longifolia compounds are readily extracted in water although none of these are active against the two test bacteria. Overall, it was the extracts of the stem material that displayed greater antibacterial activity against both of the bacteria. This result is interesting because in studies that separate the stem and leaf material of the plant, it is more often the leaf material that exhibits antibacterial activity (Palombo and Semple, 2001). Although both the acetone and ethanol stem extracts produced large zones of inhibition, only the ethanolic extract was pursued for further investigation. This is due to the fact that the ethanol resulted in a higher yield of extracted compounds (Table 1).

### Minimum Inhibitory Concentration (MIC) Assays

To assess the relative potency of the active ethanolic stem extract against each bacterial species, plate-hole diffusion assays were performed to determine the MIC values. MIC assays assess the lowest concentration required of the extract to inhibit the tested bacteria. Given the semi-quantitative nature of plate-hole assays and their reliance on the diffusibility of active compounds through agar, the results can be used as an estimate of the actual MIC. Dilutions of the stem extract were made in ethanol and the lowest concentration producing a visible zone of inhibition was deemed the MIC (Table 3).

TABLE 3
Minimum inhibitory concentrations of the ethanol extract against the
cariogenic test bacteria
E. longifolia ethanolic stem extract
MIC (mg/ml)

| | |
|---|---|
| S. mutans | 5.0 |
| S. sobrinus | 5.0 |

The ethanolic stem extract had a minimum inhibitory concentration of 5 mg/ml against both S. mutans and S. sobrinus. It is difficult to make assumptions regarding the potency of the stem extract based on its MIC values because the extract is of crude nature and has not been fractionated in any way. The active compounds within the extract may only contribute a small amount of weight to the extract whereas the majority may be comprised of inactive components; this would increase the MIC value. Nonetheless, comparisons between plant extracts based on their MIC values are considered standard procedure. Cos et al. (2006) have suggested the use of strict criteria when assessing the relative potency of extracts and phytochemicals. They have proposed that only extracts with MIC values of ≦0.1 mg/ml and phytochemicals with MIC values of ≦20 μg/ml can be considered useful for the development of products for application against oral infections. However, these criteria are the concluded suggestion of one published investigation and therefore represent only a guideline when screening plant extracts. For example, an extract of Hydrastis canadensis has been included in the formulation of a number of oral rinses and toothpastes on the US market despite showing an MIC value of only 0.25 mg/ml (Hwang et al., 2003). Furthermore, a crude extract with a relatively high MIC value may contain an active phytochemical with high potency. For example, the ethanolic extract of Piper cubeba was found to have an MIC as high as 2 mg/ml against a selection of Streptococcus species, but the isolated active compound, berberine, showed an MIC of only 13-20 μg/ml (Hu et al., 2000). The minimum inhibitory concentration of the stem extract against S. mutans and S. sobrinus is not excessively high considering that it is a crude extract resulting from a one-step extraction process. If time permitted, the extraction method could have been optimized and additional separation techniques could have been applied which most likely would have decreased the MIC value.

### Time-Kill Assay

Time-kill assays were performed so that the killing kinetics of the stem extract could be observed over a 2-hour period. Whilst the agar diffusion methods provide an end-time assessment of the extract's potency, the time-kill assays provide a dynamic analysis of the decline in viable bacteria cells. The concentration of the stem extract used in these assays was twice the MIC—10 mg/ml. This was an estimation of the lowest concentration of extract that was lethal to the bacterial cells (MBC), rather than simply preventing growth. The estimation was based on a study that noted that MBC values were commonly twice the MIC values (Furiga et al., 2008). Ideally, the MBC of an extract is determined experimentally, however the results of these analyses were inconclusive.

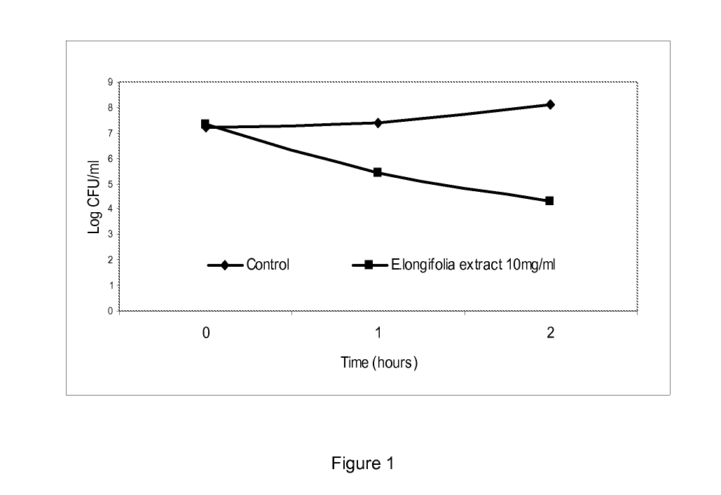

An S. mutans culture was incubated in the presence of 10 mg/ml of stem extract and samples were taken at T=0, 1 and 2 hours for a viable count, to determine the decline in viable cells (FIG. 1). A sample from the same S. mutans culture was incubated without addition of stem extract to observe a control growth curve. The extract exhibited a significant reduction in viable cells compared with the control after 1 hour (p<0.01). The stem extract exhibited bactericidal activity against S. mutans, causing a gradual decline in the number of viable cells (approximately 3.0 log units) in the test broth over 2 hours. If the extract was only capable of inhibiting the growth of the bacteria, the number of viable cells (colony forming units) would remain relatively stable in comparison with the control curve.

Although the extract does not cause a complete elimination of viable cells, the reduction is still considerable when compared with other time-kill assays in the literature. For example, Alviano et al. (2008) reported an approximate 1.8-1.5 log reduction in viable S. mutans cells over 2 hours by aqueous Cocos nucifera and Caesalpinia pyramidalis. Another extract tested in this study, from Ziziphus joazeiro, did not result in any reduction in the viable cell number despite its use in commercial dentifrices. All extracts in this study were used at a concentration of 16 mg/ml.

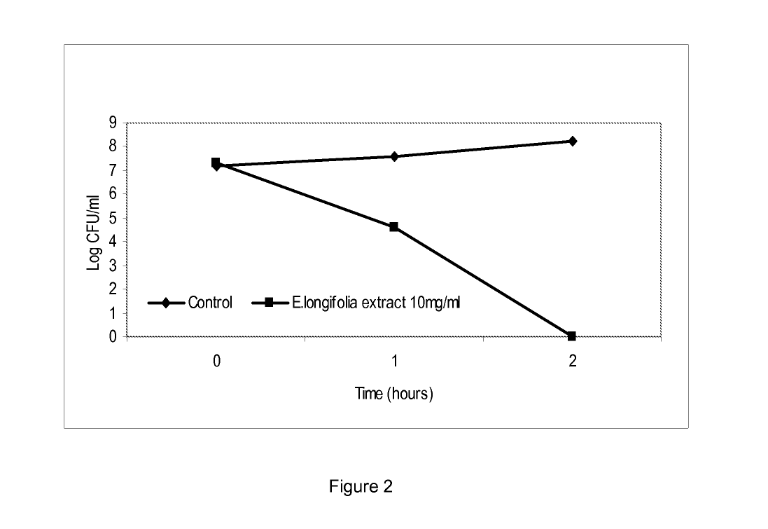

The stem extract appeared to be more potent against S. sobrinus in a 2-hour period, displaying complete elimination of viable cells (FIG. 2). The extract exhibited a significant reduction in viable cells compared with the control after 1 hour (p<0.01).

### pH Assay

Acid production by both S. mutans and S. sobrinus plays an important role in the pathology of dental caries. Lactic acid is produced through the metabolism of dietary sucrose and causes demineralization of the protective tooth enamel, leading to a carious lesion.

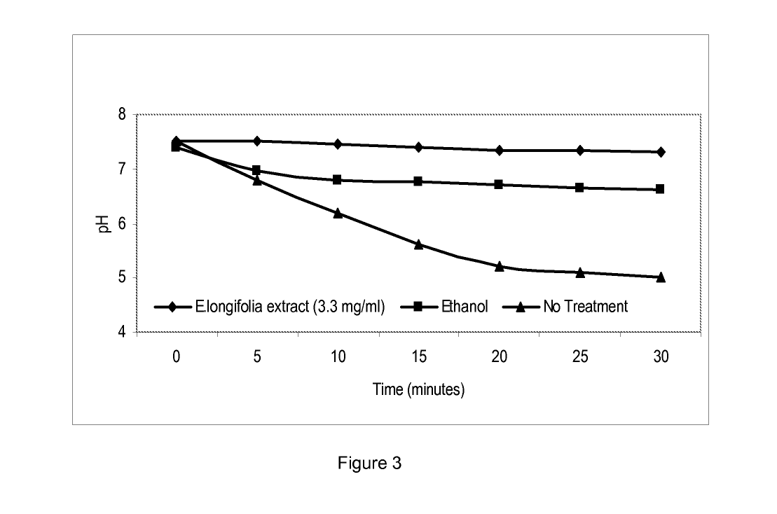

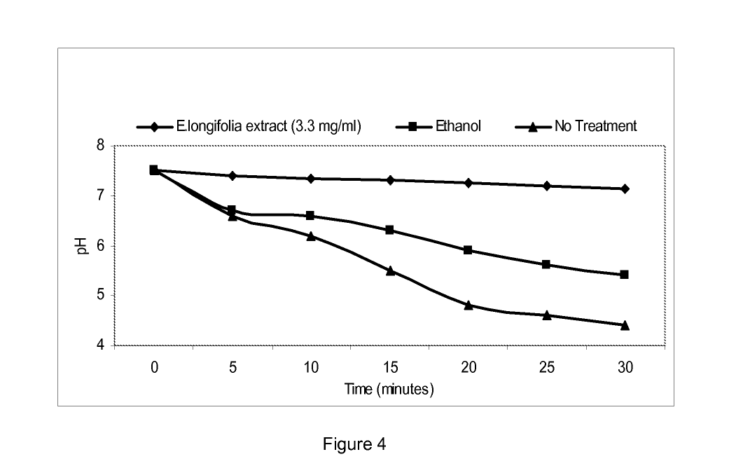

S. mutans and S. sobrinus were incubated in a 1% glucose salt solution to determine whether sub-MIC stem extract (3.3 mg/ml) was capable of reducing acid production. The pH of the solution was measured at 5-minute intervals for 30 minutes and compared with values obtained from a solvent control (ethanol) and a “no treatment” control (FIGS. 3 and 4). In both FIGS. 3 and 4, the extract exhibited a significant reduction in pH drop compared with both the ethanol control and the “no treatment” control after 5 minutes (p<0.05). The stem extract was present at a sub-MIC concentration, which means that it is not inhibiting the growth of the bacteria. Instead, the reduction in acid production suggests that the extract is affecting the bacteria's metabolism of glucose. The viability of the tested bacteria was confirmed by taking a sample from the reaction tube and successfully growing it on BHI agar.

The solvent control was performed to ensure that any conclusions made about the activity of the extract were indeed attributed to the extract and not its ethanol content. In both the S. mutans and S. sobrinus assays, the addition of 166 μL of ethanol resulted in a reduction of acid produced. However, the pH values remain more stable with addition of the extract and its increased inhibition of acid production is statistically significant, especially in the S. sobrinus assay (P<0.05).

### Antibacterial Activity Against Salivary Bacteria

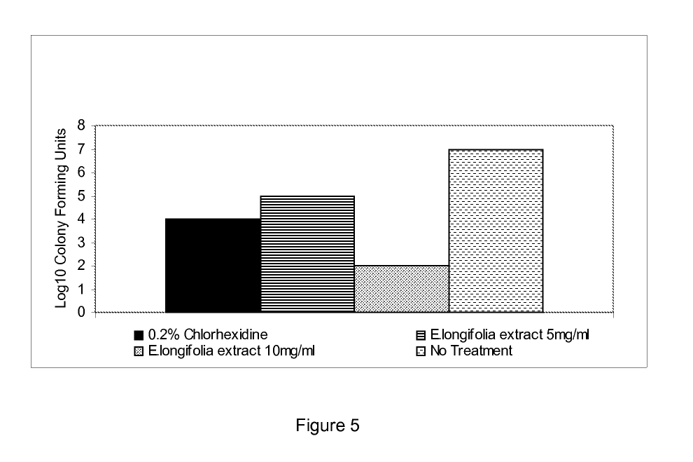

It is possible that components within saliva can interact with active compounds within a plant extract and render it inactive against its target bacteria. Because of this, it is important to assess the antibacterial activity of the plant extract in the presence of saliva. This was achieved by incubating saliva samples in the presence of the stem extract (5 mg/ml and 10 mg/ml) and chlorhexidine (0.2 mg/ml) and performing a viable count after 1 hour (FIG. 5). One sample of saliva was incubated without addition of any treatment, to serve as a control. The four values were significantly different from each other (p<0.05). Both concentrations of stem extract caused a reduction in viable salivary bacteria. This suggests that the extract remains active in the presence of saliva.

### Artificial Biofilm Assays

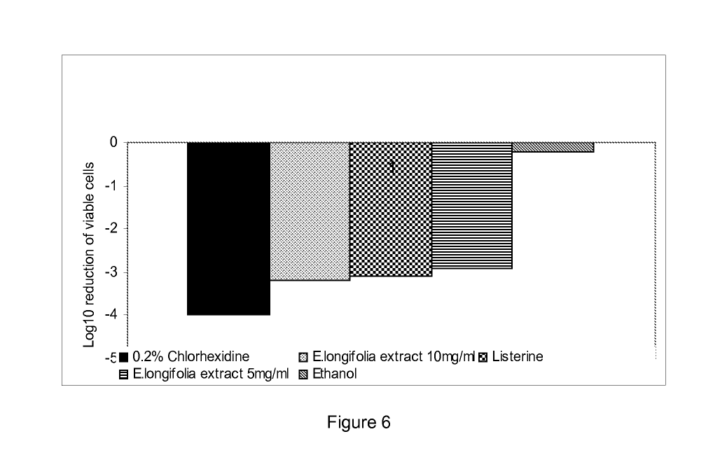

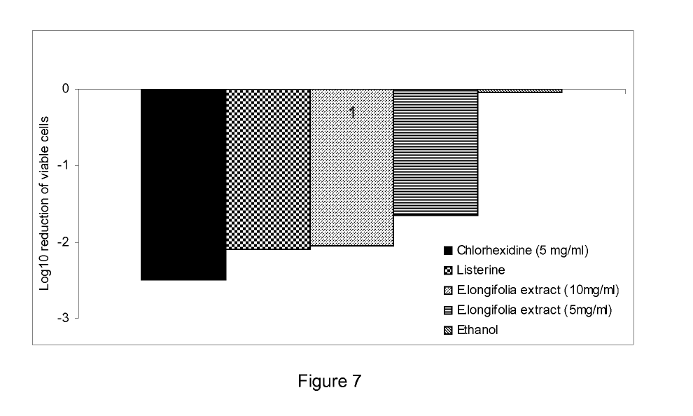

The attachment of pathogenic bacteria to the tooth surface, and the formation of a biofilm structure, is a key element in the formation of dental caries. An assessment of the activity of the stem extract on a bacterial biofilm was achieved by reproducing in vitro biofilms with human saliva and S. mutans. The artificial biofilms were grown on membrane filters and placed into 3 ml of stem extract (5 mg/ml and 10 mg/ml), ListerineR, chlorhexidine, ethanol and water. After incubation for 1 hour, the number of bacteria cells remaining on the membrane filters was determined (FIGS. 6 and 7).

FIG. 6 shows the results of a salivary bacteria artificial biofilm assay. All four treatments showed a significant reduction in viable cells (p<0.01) compared with the ethanol and water controls. The four treatments are not significantly different from each other (p>0.05) and the ethanol control did not cause a significant reduction compared with the water control (p>0.05).

FIG. 7 shows the results of a S. mutans artificial biofilm assay. All four treatments showed a significant reduction in viable cells (p<0.01) compared with the ethanol and water controls. The four treatments are not significantly different from each other (p>0.05) and the ethanol control did not cause a significant reduction compared with the water control (p>0.05).

In both the salivary bacteria and S. mutans assays, the chlorhexidine (5 mg/ml) produced the greatest reduction in viable biofilm bacteria. However, the commercial product Listerine and the two stem extracts were able to also significantly reduce the viable count and were not significantly different from the chlorhexidine reduction. It was not surprising that Listerine had such an effect, as it is marketed as an antiseptic mouth rinse that targets bacteria in the plaque biofilm within a recommended treatment time of only 0.5 minutes. The treatment time in these assays was 60 minutes.

The significant reduction in viable biofilm cells by the stem extract is important because biofilm-associated bacteria are more capable of tolerating the presence of antimicrobial agents (Djordjevic et al., 2002). The results from both assays suggest that the extract is capable of detaching the cells from the biofilm and/or killing cells that remain attached. This first point is important as it may be preferential that an active agent is anti-adhesive rather than bactericidal in order to reduce the development of resistant strains (Duarte et al., 2006).

### Scanning Electron Microscopy (SEM) Analysis

Salivary bacteria biofilms and S. mutans biofilms were grown on membrane filters as described in the section entitled “Artificial biofilm assays”. SEM was performed to determine if there was any evidence of a biofilm on the filters.

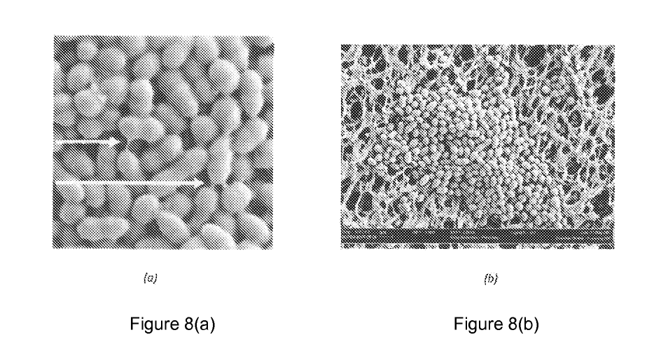

FIG. 8(a) shows an SEM image of a dense cell population within the salivary bacteria sample. There appears to be an extracellular substance between some of the cells which may be a polysaccharide involved in the early attachment process of biofilm formation. FIG. 8(b) shows a dense cluster of S. mutans cells. Although an extracellular substance could not be observed on this filter membrane, the cell population shows depth and is strongly attached as both filters were rinsed with sterile water prior to SEM analysis. Further analysis of additional membranes may have produced evidence of biofilm formation.

### Inhibition of Attachment

A standard method for assessing an extract's ability to inhibit biofilm formation is the microtiter plate procedure. Bacteria is grown in the plate's wells and allowed to adhere to the sides. Quantification of biofilm accumulation involves staining the attached cells with crystal violet and measuring the optical density of each sample using a plate reader (Rasooli et al., 2008; Djordjevic et al., 2002). This method was initially performed, however inconclusive results were obtained. This is because the stem extracts changes from a brown to purple colour when warmed in the presence of BHI broth due perhaps to the presence of anthocyanidins. The purple colour was very similar to the crystal violet and interfered with the plate reader values. The assay used in this study was based on a beaker-wire test performed by Kang et al. (2008), which evaluated S. mutans accumulation on stainless steel wire in the presence of a treatment. Initially, the Kang et al. method was followed but inconclusive results were obtained. The method relied on the accumulation of S. mutans to be large enough to be quantified by weight.

The published study obtained a mean plaque weight of 198.5 mg whereas replication of this method could only produce a mean weight of 6.3 mg. Also, the stem extract attached to the wire and could not be removed with rinsing, adding to the weight. Due to these limitations, the beaker-wire test was modified into a more suitable method. To increase the number of S. mutans cells involved in attachment, a membrane filter was used instead of stainless steel wire.

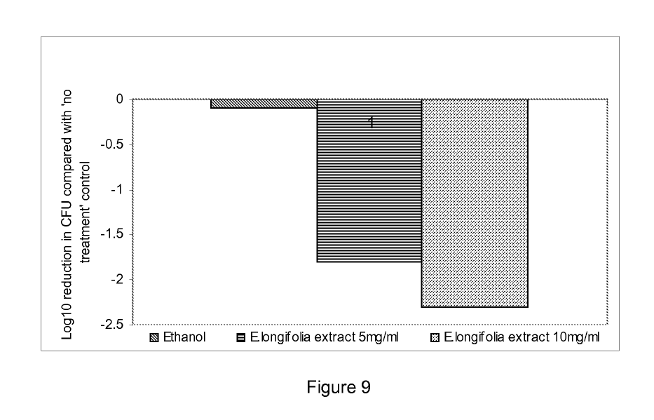

Quantification of attached cells was determined by vortexing the membranes in solution and performing a viable count of detached cells. The effects of the stems extract (5 mg/ml and 10 mg/ml) and ethanol was compared with a “no treatment” control (FIG. 9). FIG. 9 shows inhibition of S. mutans attachment to 0.22 μm membrane filters. The stem extracts both show a significant reduction in viable cells (p<0.05) compared with the control. The stem extracts are not significantly different from each other (p>0.05) and the ethanol control did not cause a significant reduction compared with the “no treatment” control (p>0.05). As attachment of cariogenic bacteria to teeth is an important feature of dental caries pathology, significant inhibition of this characteristic would be an ideal property of a caries preventative treatment. The stem extract (at both concentrations) was able to significantly reduce the number of S. mutans that attached to the membrane filter.

### Preliminary Phytochemical Analysis—Microscale Column Chromatography

A 150 μL sample of stem extract (100 mg/ml) was separated in a glass Pasteur pipette containing silica gel. The mobile phase was initially hexane: ethanol, 9:1, and was then changed to hexane: ethanol, 6:4 after the addition of approximately 3.4 ml. Fractions were collected as separate coloured bands passed through the column. Ten fractions were collected, dried, and re-dissolved in ethanol to a concentration of 100 mg/ml. All fractions were screened for antibacterial activity against S. mutans and S. sobrinus by plate-hole diffusion.

TABLE 4
Summary of fractions that showed antibacterial activity (zone of
inhibition >6 mm).

| | | Zone of |
|---|---|---|
| Fraction | Colour | Inhibition |
| 3 | Yellow | 8.0 +/− 0.6 |
| 6 | Pink | 7.5 +/− 0.6 |
| 7 | Orange | 11.5 +/− 0.9 |

Values represent the combined mean of the growth inhibition diameter plus SD from two S. mutans and two S. sobrinus assays (N=4).

Only three fractions were capable of inhibiting the growth of S. mutans and S. sobrinus. Their zones of inhibited growth were smaller than those produced from the crude extract (Table 2), which suggests that the active compounds present in the extract may have been separated and eluted in the three different fractions. Although fraction 7 produced the greatest zone of inhibition, it was very small and was exhausted in the plate-hole assays. Therefore, fraction 3 was used for further phytochemical analysis.

### Thin Layer Chromatography (TLC) and Bioautography

A preliminary investigation into the identity of the active compounds within fraction 3 was undertaken. The first step involved separation of the fraction using thin layer chromatography (TLC). Three solvent systems were trialled and the toluene: ethanol, 9:1 solvent provided optimal separation of compounds in the TLC chromatogram. Comparison between this and the TLC of the crude stem extract in the same solvent system demonstrated that the fraction contained far fewer coloured bands. Bioautography assays were performed on TLC plates to determine which separated band contained active compounds (Table 5).

TABLE 5
Rf values of areas on TLC plates producing zones of growth inhibition
in bioautography assays.

| | Crude | |
|---|---|---|
| | extract | Fraction |
| S. mutans | 0.00-0.42 | 0.00 |
| | | 0.32 |
| | | 0.44 |
| S. sobrinus | 0.00-0.34 | 0.00 |
| | | 0.17 |
| | | 0.25 |
| | | 0.32 |

The areas of inhibition produced by the separated fraction correlate with the large zone of inhibition observed in the crude extract bioautography assays. All zones were positioned on the lower half of the silica gel TLC plate; as the mobile phase was relatively non-polar, this indicated that the active compounds were relatively polar.

### Identification of Active Compound Groups Using Spray Reagents

Four different spray reagents were used on developed TLC plates of fraction 3 to identify the compound class of the active component. Only the Folin-Ciocalteu reagent returned a positive result. This indicated the presence of phenolic compounds in the same areas that showed antibacterial activity in the bioautography assays. Analysis of the TLC plate under UV 254 nm light showed dark spots in the same areas that reacted with the Folin-Ciocalteu reagent. Phenolic compounds are able to quench fluorescence at this wavelength, resulting in dark spots. However, other structures are also known to cause this effect (Harbourne, 1973). Analysis of the TLC plate under UV 366 nm revealed bright blue fluorescence at most of the areas indicated by the Folin-Ciocalteu reagent. Flavonoids are phenolic structures and are known to produce fluorescent blue, purple and green at this wavelength. However, the aluminium chloride spray reagent for detection of flavonoids was negative. This reagent results in fluorescent yellow being produced where flavonoids are present. It may have been that this colour was difficult to see or that the reagent did not react as indicated. Using these results, a preliminary estimation as to the active compounds' class was a polyphenolic compound. Furthermore, despite the AlCl3 spray results, it is possible that the compounds are flavonoids as these are ubiquitous in plants. These compounds are also relatively polar, which corresponds to the positions of growth inhibition in the bioautography assays. Flavonoids have been found to be effective antimicrobial substances in vitro against a wide array of microorganisms. It is thought that their activity is related to their ability to complex with extracellular and soluble proteins and to complex with bacterial cell walls (Cowan 1999).

### Preliminary GC-MS Analysis of Fraction 3

GC-MS analysis was initially performed to identify some prominent compounds within fraction 3 of the stem extract, and to determine approximately how many compounds were in the fraction. However, only small peaks were produced on the chromatogram and these were not sufficient to confidently indicate the number of compounds in the sample. Furthermore, none of the compounds could be confidently identified using the existing GC-MS library.

### CONCLUSIONS

A sample of the traditional medicinal plant Eremophila longifolia was extracted in three different polar solvents and screened for antibacterial activity against the cariogenic bacteria Streptococcus mutans and S. sobrinus. The ethanolic extract of the stem material was investigated further as it displayed large zones of inhibition in agar diffusion methods and was produced in relatively high yield. Time-kill assays showed that the stem extract, at a concentration of 10 mg/ml, was able to eliminate all viable S. sobrinus cells within a 2-hour period. At 3.3 mg/ml, it was able to inhibit acid production by both of the test bacteria without killing them. This result is important in terms of anti-cariogenic activity as it is the acid produced by cariogenic bacteria that causes demineralisation of tooth enamel and dentin, leading to a carious lesion. Artificial biofilm assays were also performed to determine whether the extract was capable of remaining active in the presence of saliva, affecting bacteria within a biofilm or inhibiting initial attachment of bacteria to a surface. In all these assays, the extract showed a statistically significant difference compared with a negative control.

Preliminary phytochemical analysis of the stem extract was also performed in this study. Separation of the extract by microscale column chromatography produced three fractions with antibacterial activity. One fraction was analysed by bioautography and displayed three to four distinct areas of activity against S. mutans and S. sobrinus. Investigations using spray reagents and UV analysis on the TLC plates suggested that the active compounds were phenolics, and possibly flavonoids.

### Example 2

### In Vitro Activity of Emubush Extract Against Gram-Positive Bacteria and Gram-Negative Bacteria

### Materials and Methods

A study was carried out to determine the minimum inhibitory concentration (MIC) for Emubush extract and a comparator against a panel of isolates using Clinical and Laboratory Standards Institute (CLSI) broth methodology.

### Test Isolates

The extract was tested against a panel of Gram-positive and Gram-negative bacteria. All isolates are from the Quotient Bioresearch Ltd., Microbiology collection.

### Test Material

Levofloxacin was used as a comparator antibacterial.

The Emubush extract was prepared by dissolving 95.2 mg in ethanol. The solvent was then evaporated off and the material re-dissolved in ethanol at room temperature.

### Minimum Inhibitory Concentration (MIC) Determination

MIC was determined by microbroth dilution following CLSI methodology. [CLSI Methods for Dilution Antimicrobial Susceptibility Tests for Bacteria That Grow Aerobically; Approved Standard-Eighth Edition. CLSI Document M07-A8. CLSI, Wayne, Pa. 19087-1898, USA, 2009].

### Results

A listing of MIC data is shown in Tables 6 and 7. The Emubush extract was considered to be effective where an MIC of 64 mg/L or less was achieved.

TABLE 6
Gram-positive MIC results.

| | | Emubush | |
|---|---|---|---|
| | | Extract | Levofloxacin |
| Isolate | Strain | (mg/L) | (mg/L) |
| GP1 | Staphylococcus aureus ATCC | 8 | 0.25 |
| | 29213 - antibiotic-susceptible | | |
| | type strain. | | |
| GP3 | Staphylococcus aureus ATCC | 8 | 0.12 |
| | 43300 - methicillin-resistant type | | |
| | strain. | | |
| GP4 | Staphylococcus aureus - methicillin- | 8 | 0.12 |
| | resistant clinical isolate. | | |
| GP7 | Staphylococcus epidermidis - | 2 | 0.12 |
| | antibiotic susceptible clinical | | |
| | isolate. | | |
| GP8 | Staphylococcus epidermidis - | 4 | 0.12 |
| | methicillin-resistant clinical | | |
| | isolate. | | |
| GP24 | Streptococcus pneumoniae - multi- | 64 | 1 |
| | drug resistant clinical isolate. | | |
| GP59 | Streptococcus pyogenes - antibiotic- | 64 | 0.5 |
| | susceptible clinical isolate. | | |

TABLE 7
Gram-negative MIC results.

| | | Emubush | |
|---|---|---|---|
| | | Extract | Levofloxacin |
| Isolate | Strain | (mg/L) | (mg/L) |
| GN08 | Serratia marcescens - antibiotic- | 64 | 0.06 |
| | susceptible clinical isolate | | |
| GN09 | Serratia marcescens - multi-drug | 64 | 2 |
| | resistant clinical isolate | | |
| GN11 | Pseudomonas aeruginosa - multi- | 64 | 4 |
| | drug resistant clinical isolate | | |
| GN12 | Stenotrophomonas maltophilia - | 32 | 0.12 |
| | antibiotic-susceptible clinical isolate | | |
| GN13 | Stenotrophomonas maltophila - | 32 | 2 |
| | antibiotic-resistant clinical isolate | | |
| GN14 | Burkholderia cepacia - antibiotic- | 32 | 1 |
| | susceptible clinical isolate | | |

### CONCLUSION

The Emubush extract was shown to have activity against Staphylococcus aureus, Staphylococcus epidermidis, Streptococcus pneumoniae, Streptococcus pyogenes, Serratia marcescens, Pseudomonas aeruginosa, Stenotrophomonas maltophilia and Burkholderia cepacia.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A method for inhibiting growth of bacteria, the method comprising the step of:
administering to a region in need of bacterial growth inhibition an anti-bacterially effective amount of a composition comprising an extract from the plant Eremophila longifolia.

2. The method as claimed in claim 1 wherein the composition comprising the extract is administered to a mammal.

3. The method as claimed in claim 2 wherein the composition is administered to an oral cavity of the mammal.

4. The method as claimed in claim 3 wherein the method is a method for reducing formation of lactic acid, inhibiting formation and attachment of a plaque biofilm and/or inhibiting tooth decay.

5-6. (canceled)

7. The method as claimed in claim 2 wherein the method is a method for inhibiting the production of acid by Streptococcus sobrinus and/or Streptococcus mutans.

8. (canceled)

9. The method as claimed in claim 3 wherein the composition is selected from the group consisting of a mouth wash, toothpaste, chewing gum, lozenge and powder.

10. The method as claimed in claim 1 wherein the method is an ex vivo method.

11. The method as claimed in claim 10 wherein the method is a method for inhibiting the attachment and growth of a bacterial biofilm.

12. The method as claimed in claim 10 wherein the region in need of bacterial growth inhibition is selected from the group consisting of a surface of a medical device, a water system, a ventilation system, a plumbing system, an air conditioner, a humidifier and a hot tub.

13. (canceled)

14. The method as claimed in claim 1 wherein the method further comprises the step of administering one or more additional anti-bacterial agents.

15. A composition comprising an extract from the plant Eremophila longifolia for use in inhibiting bacteria.

16. The composition as claimed in claim 15 wherein the composition is selected from the group consisting of a mouth wash, toothpaste, chewing gum, lozenge and powder.

17-18. (canceled)

19. The method as claimed in claim 1 wherein the bacteria comprise at least one bacteria selected from the group consisting of Staphylococcus aureus, Staphylococcus epidermidis, Streptococcus pneumoniae, Streptococcus pyogenes, Serratia marcescens, Pseudomonas aeruginosa, Stenotrophomonas maltophilia, Legionella pneumophila and Burkholderia cepacia.

20. (canceled)

21. A method for the treatment and/or prophylaxis of cariogenesis, halitosis, gingivitis and periodontitis in a mammal, the method comprising the steps of:
providing a therapeutically effective amount of a composition comprising an extract from the plant Eremophila longifolia; and
administering the composition to the mammal.

22. The method as claimed in claim 21 wherein the composition is administered to an oral cavity.

23. (canceled)

24. The method as claimed in claim 21 wherein the composition is selected from the group consisting of a mouth wash, toothpaste, chewing gum, lozenge and powder.

25. The method as claimed in claim 21 wherein the method further comprises the step of administering one or more additional anti-bacterial agents to the mammal.

26-28. (canceled)

29. A method for the treatment and/or prophylaxis in a subject of one or more of the conditions selected from the group consisting of Legionnaires' disease, sepsis, endocarditis, skin infections, impetigo, cellulitis folliculitis, scalded skin syndrome, pneumonia, meningitis, osteomyelitis, toxic shock syndrome, mastitis, acute sinusitis, otitis media, bacteremia, septic arthritis, peritonitis, pericarditis, brain abscess, Pharyngitis, erysipelas, cellulitis, necrotizing fasciitis, rheumatic fever, glomerulonephritis, obsessive compulsive disorder, tic disorders, urinary tract infections, respiratory tract infections, conjunctivitis, keratitis, endophthalmitis, tear duct infections, teeth staining, white pox disease, viral falcerie disease, Septicaemia, necrotising enterocolitis, haemorrhage and necrosis, hot tub rash, blood stream infections and cystic fibrosis, wherein the method comprises the steps of:
providing a therapeutically effective amount of a composition comprising an extract from the plant Eremophila longifolia; and
administering the composition to the subject.

30. The method as claimed in claim 29 wherein the method further comprises the step of administering one or more additional anti-bacterial agents to the subject.

31-32. (canceled)

33. The method as claimed in claim 1 wherein the plant extract is derived from the stem, leaves, roots, branches, fruit or flower of Eremophila longifolia.

34. The method as claimed in claim 33 wherein the plant extract is derived from the stem of Eremophila longifolia.

35. The method as claimed in claim 1 wherein the extract is from Eremophila longifolia of the types cultivated in New South Wales and/or the Northern Territory of Australia.

36. The method as claimed in claim 1 wherein the plant extract is an ethanolic extract.
